(19) United States
(12) Patent Application Publication
(10) Pub. No.: US 2026/0207267 A1
(43) Pub. Date: Jul. 23, 2026

(54) Shape Sensing Fiber Optic Tip Protection Systems and Devices

(71) Applicant: Bard Access Systems, Inc., Salt Lake City, UT (US)

(72) Inventors: Anthony K. Misener, Bountiful, UT (US); Steffan Sowards, Salt Lake City, UT (US); William Robert McLaughlin, Bountiful, UT (US)

(21) Appl. No.: 19/568,573

(22) Filed: Mar. 16, 2026

Related U.S. Application Data
(63) Continuation of application No. 17/849,447, filed on Jun. 24, 2022, now Pat. No. 12,575,893.

(51) Int. Cl. A61B 34/20; A61B 1/07; A61B 5/06; G02B 6/02; G02B 6/36

(52) U.S. Cl. CPC A61B 34/20; A61B 1/07; A61B 5/065; G02B 6/02057; G02B 6/02076; A61B 2034/2046; A61B 2034/2061; G02B 6/3624

(57) ABSTRACT

A fiber-optic assembly for insertion in a patient, includes a shape sensing optical fiber and a mechanical layer surrounding a distal section of the optical fiber. The distal section includes an optical fiber tip and an expansion configured to inhibit damage of the optical fiber tip. The expansion includes a proximal end proximal of the optical fiber tip, a distal end distal of the optical fiber tip, and a diameter greater than a diameter of the shape sensing optical fiber. The optical fiber can include a plurality of optical fiber cores. One or more of the plurality of optical fiber cores can include a plurality of sensors distributed along at least the distal portion of the optical fiber configured to project reflected light signals proximally along the optical fiber to indicate a shape of the optical fiber.

### PRIORITY

This application is a continuation of U.S. Patent Application No. 17/849,447, filed June 24, 2022, now U.S. Patent No. 12,575,893, which is incorporated by reference in its entirety into this application.

### BACKGROUND

Shape sensing optical fibers include shape sensing regions along the optical fiber. Sensors, such as fiber Bragg gratings, generate reflected light signals. In some instances, the sensing regions may extend to the proximity of the distal tip of the optical fiber. In some instances, the distal tip portion may become damaged during use causing a reduction in shape sensing capability. In some instances, the damage may include the end face of the optical fiber resulting in back scattering of light signals that may also cause a reduction in shape sensing capability.

Disclosed herein are fiber-optic tip protection devices and systems that address the foregoing.

### SUMMARY

Briefly summarized, disclosed herein is a fiber-optic assembly for insertion within a patient body, that according to some embodiments, includes an optical fiber having a distal tip and a distal portion extending proximally away from the distal tip and a secondary mechanical layer extending around a circumference of the optical fiber along at least the distal portion of the optical fiber. The optical fiber includes a distal tip section configured to inhibit damage of the distal tip during use of the optical fiber, where the distal tip section (i) encompasses the distal tip, (ii) is disposed within the secondary mechanical layer, and (iii) includes a portion of the optical fiber disposed within a circuitous path, or an expansion of the optical fiber.

In some embodiments, the optical fiber includes a number of optical fiber cores, and one or more of the number of the optical fiber cores includes a number of sensors distributed along at least the distal portion of the optical fiber, where the sensors are configured to project reflected light signals proximally along the optical fiber based on a shape of the optical fiber.

In some embodiments, the secondary mechanical layer extends distally beyond the distal tip. The secondary mechanical layer may include a coil, tube or a combination of the coil and the tube.

In some embodiments, the assembly may further include a substance disposed within the secondary mechanical layer, where the substance defines a mechanical buffer between the optical fiber and the secondary mechanical layer. The substance may include an elastomeric material or a fluid. The secondary mechanical layer may include a closed distal end to facilitate containing the substance within the secondary mechanical layer. In some embodiments, the secondary mechanical layer may be coupled with the optical fiber so as to inhibit longitudinal displacement of the optical fiber with respect to the secondary mechanical layer.

In some embodiments, the secondary mechanical layer defines a circuitous path for disposition of the distal section therein. In such embodiments, the wherein the circuitous path may define a number of curved sections that include one or more of a spiral, a coil, a serpentine shape, a loop, a J-shape, or any combination thereof.

In some embodiments, the distal section includes an expansion (or expanded portion) defining a diameter that is greater than a diameter of the optical fiber. The expansion may be integrally formed of a material of the optical fiber or the expansion may be formed of an elastomeric material attached to the optical fiber. In some embodiments, the expansion is electrically conductive and, in some embodiments, the secondary mechanical layer is electrically conductive. In some embodiments, the expansion is electrically coupled with the secondary mechanical layer, and in some embodiments, the expansion extends beyond an open end of the secondary mechanical layer.

Also disclosed herein in is an elongate medical device, that according to some embodiments, includes an optical fiber assembly coupled with any one of a catheter, a stylet, a probe, or a guidewire. The optical fiber assembly includes an optical fiber defining a distal tip and a distal portion extending proximally away from the distal tip. The optical fiber assembly further includes a secondary mechanical layer extending around a circumference of the optical fiber along at least the distal portion of the optical fiber. The optical fiber includes a distal tip section configured to inhibit damage of the distal tip during use of the medical device assembly, where the distal tip section (i) encompasses the distal tip, (ii) is disposed within the secondary mechanical layer, and (iii) includes a portion of the optical fiber disposed within a circuitous path, or an expansion of the optical fiber.

In some embodiments, the optical fiber includes a number of optical fiber cores, where one or more of the number of the optical fiber cores includes a number of sensors distributed along at least a portion of the optical fiber, and the sensors are configured to project reflected light signals proximally along the optical fiber based on a shape of the at least a portion of the optical fiber.

In some embodiments, at least a subset of the number of sensors are distributed along the distal portion, where the distal portion includes the distal tip.

In some embodiments, the elongate medical device further includes a number of electrically conductive elements extending along the optical fiber, and the distal tip section includes an expansion formed of an electrically conductive material to define an electrode of the medical device, and where the electrode is coupled with at least one of the number electrically conductive elements.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 is an illustration of a first embodiment of a fiber optic assembly, in accordance with some embodiments;

FIG. 2 is an illustration of a second embodiment of a fiber-optic assembly, in accordance with some embodiments; and

FIG. 3 is an illustration of a third embodiment of a fiber-optic assembly, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The terms “proximal” and “distal” refer to opposite directions of a medical device, including the devices disclosed herein. As used herein, the distal portion of a medical device is the portion nearest a patient during use. For example, the distal portion is defined as the portion of the device nearest to, or furthest inserted into, the patient. By way of further example, a component or feature extending proximally or in a proximal direction, extends away from the patient or in a direction toward an exterior of the patient.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

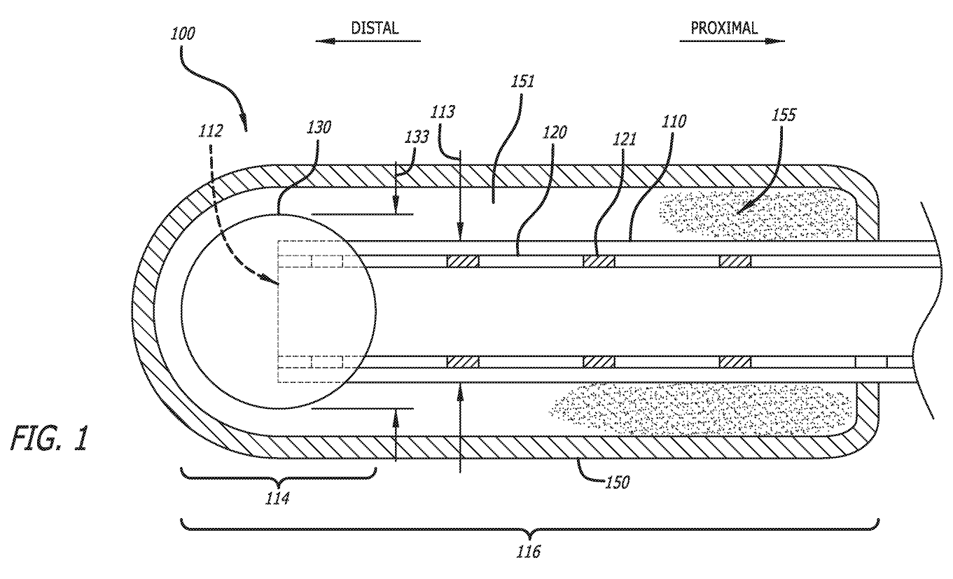

FIG. 1–3 illustrate different embodiments of a fiber-optic assembly, where the fiber optic assembly includes components and features that define a functionality of a fiber-optic tip protection system. Any of the fiber-optic assembly embodiments described herein below may be coupled with, incorporated into, or otherwise employed with another medical device to define a medical device assembly. In some embodiments, the other medical device may be any one of a catheter, a stylet, a probe, or a guidewire.

FIG. 1 is an illustration of a first embodiment of a fiber-optic assembly, in accordance with some embodiments. The fiber optic assembly generally includes components coupled with and/or integrally formed with an optical fiber along a distal portion of the optical fiber. In accordance with a first embodiment, the fiber-optic assembly (assembly) 100 includes an optical fiber 110 and a secondary mechanical layer 150 extending around a circumference of the optical fiber 110. The optical fiber 110 defines a distal tip 112, where the distal tip 112 defines the distal most end of at least a subset of optical operations of the optical fiber 110. In other words, at least one optical operation of the optical fiber 110 extends to the distal tip 112.

The optical fiber 110 may optionally be configured for shape sensing. In the illustrated embodiment, the optical fiber 110 includes a number (e.g., 1, 2, 3, 4, or more) of optical fiber cores 120, extending along a length of the optical fiber 110, that include a plurality of sensors (e.g., fiber Bragg gratings) 121 configured to project reflected light signals proximally along the optical fiber 110 based on a shape of the optical fiber 110.

The optical fiber 110 includes an expansion 130 disposed along a distal section 114 of the optical fiber 110 where the distal section 114 encompasses the distal tip 112. In the illustrated embodiment, the distal section 114 extends both proximally and distally in relation to the distal tip 112. However, in other embodiments, the distal section 114 may extend only proximally or only distally in relation to the distal tip 112.

The expansion 130 is configured to inhibit/prevent damage to the distal tip 112 during use of the optical fiber 110. The expansion 130 (or expanded portion) defines a diameter 133 that is greater than a diameter 113 of the optical fiber 110. The expansion 130 is formed of a material included with the optical fiber 110. In the illustrated embodiment, the expansion 130 includes a bulbous shape. In other embodiments, the expansion 130 may include other shapes, such as cylindrical, spherical, or teardrop shape, for example.

The secondary mechanical layer 150 is also configured to inhibit/prevent damage to the distal tip 112 during use of the optical fiber 110 in combination with the expansion 130. The secondary mechanical layer 150 may define an enhanced structural integrity to the optical fiber 110, such as a resistance to crushing, abrasion, or kinking, for example. In the illustrated embodiment, the secondary mechanical layer 150 is formed of a tube. In other embodiments, secondary mechanical layer 150 may be formed of a coil or a combination of the tube and the coil.

The secondary mechanical layer 150 may be disposed along a distal portion 116 of the optical fiber 110. The distal portion 116 generally extends proximally away from the distal tip 112. In some embodiments, the distal portion 116 may extend proximally away from the distal tip 112 a distance of about 1 mm, 2 mm, 4 mm, 8 mm, 16 mm, or more. The secondary mechanical layer 150 may also extended distally away from the distal tip 112. As such, the distal section 114 and the distal tip 112 may be included within the distal portion 116. In the illustrated embodiment, the secondary mechanical layer 150 includes a closed distal end. However, in other embodiments, the secondary mechanical layer 150 may include an open distal end.

The secondary mechanical layer 150 may be formed of a plastic, elastomeric, or metallic material. In some embodiments, the secondary mechanical layer 150 may be structurally formed defining a lumen 151 within which the optical fiber 110 may be subsequently inserted. In other embodiments, the secondary mechanical layer 150 may be formed onto the optical fiber 110, such as sprayed onto, over-molded onto, or otherwise applied to the optical fiber 110. In some embodiments, the secondary mechanical layer 150 may be attached to the optical fiber 110 so as to prevent longitudinal displacement of the secondary mechanical layer 150 with respect to the optical fiber 110. In some embodiments, the secondary mechanical layer 150, or more specifically a wall of the secondary mechanical layer 150, may define a fluid barrier. In some embodiments, the structure of the secondary mechanical layer 150 may be defined so as to allow the optical fiber 110 to flex/bend in accordance with the shape sensing operation of the optical fiber 110.

In some embodiments, the assembly 100 may include a buffering substance 155 disposed within the lumen 151 so as to occupy the annular space between the optical fiber 110 and the secondary mechanical layer 150. The buffering substance 155 may further inhibit/prevent damage to the distal tip 112 during use of the optical fiber 110 in combination with the expansion 130 and the secondary mechanical layer 150. The buffering substance 155 may include an elastomeric material or a fluid.

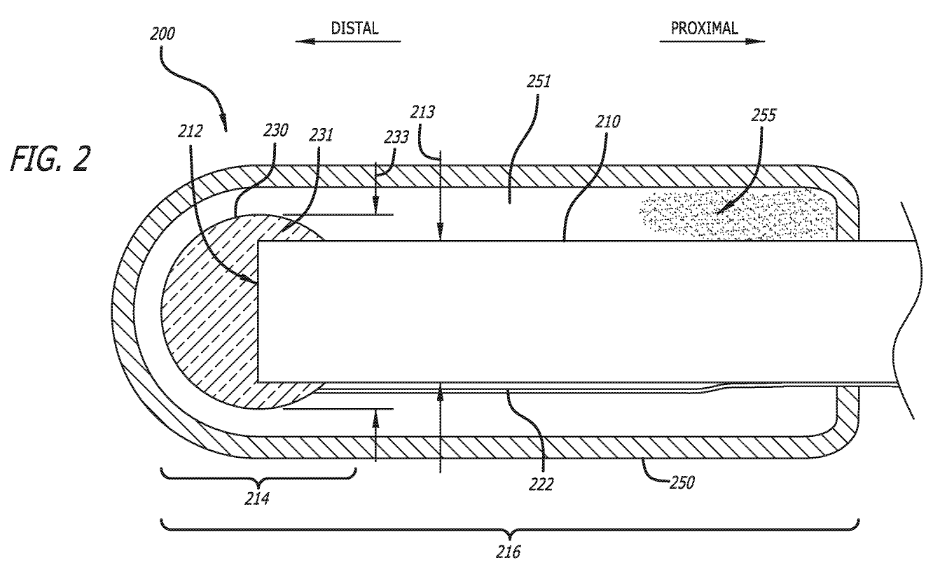

FIG. 2 is an illustration of a second embodiment of a fiber-optic assembly 200, in accordance with some embodiments, that can, in certain respects, resemble components of the fiber-optic assembly 100 described in connection with FIG. 1. It will be appreciated that all the illustrated embodiments may have analogous features. Accordingly, like features are designated with like reference numerals, with the leading digits incremented to “2.” For instance, the optical fiber is designated as “110” in FIG. 1, and an analogous optical fiber is designated as “210” in FIG. 2. Relevant disclosure set forth above regarding similarly identified features thus may not be repeated hereafter. Moreover, specific features of the fiber-optic assembly 100 and related components shown in FIG. 1 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the fiber-optic assembly 100. Any suitable combination of the features, and variations of the same, described with respect to the fiber-optic assembly 100 and components illustrated in FIG. 1 can be employed with the fiber-optic assembly 200 and components of FIG. 2, and vice versa. This pattern of disclosure applies equally to further embodiments depicted in subsequent figures and described hereafter.

The fiber-optic assembly (assembly) 200 includes an optical fiber 210 and a secondary mechanical layer 250 extending around a circumference of the optical fiber 210. The optical fiber 210 defines a distal tip 212, where the distal tip 212 defines the distal most end of at least a subset of optical operations of the optical fiber 210. In other words, at least one optical functionality of the optical fiber 210 extends to the distal tip 212. The optical fiber 210 may optionally be configured for shape sensing.

The optical fiber 210 includes an expansion 230 coupled with the optical fiber 210 so as to encompasses the distal tip 212, where the expansion 230 is disposed along the distal section 214 of the optical fiber 210 where the distal section 214. In the illustrated embodiment, the distal section 214 extends both proximally and distally in relation to the distal tip 212.

The expansion 230 is configured to inhibit/prevent damage to the distal tip 212 during use of the optical fiber 210. The expansion 230 (or expanded portion) defines a diameter 233 that is greater than a diameter 213 of the optical fiber 210. The expansion 230 is formed of a compliant material 231, such as an elastomeric material. Exemplary elastomeric materials may include silicone, EPDM, natural rubber, and the like. In some embodiments, the compliant material 231 may be electrically conductive and as such, the expansion 230 may define an electrode configured for detecting electrical signals within a patient body.

The secondary mechanical layer 250 is also configured to inhibit/prevent damage to the distal tip 212 during use of the optical fiber 210 in combination with the expansion 230. The secondary mechanical layer 250 may define an enhanced structural integrity to the optical fiber 210, such as a resistance to crushing, abrasion, or kinking, for example. In the illustrated embodiment, the secondary mechanical layer 250 is formed of a tube. In other embodiments, secondary mechanical layer 250 may be formed of a coil or a combination of the tube and the coil.

The secondary mechanical layer 250 may be disposed along a distal portion 216 of the optical fiber 210. The distal portion 216 generally extends proximally away from the distal tip 212. In some embodiments, the distal portion 216 may extend proximally away from the distal tip 212 a distance of about 1 mm, 2 mm, 4 mm, 8 mm, 16 mm, or more. The secondary mechanical layer 250 may also extended distally away from the distal tip 212. As such, the distal section 214 and the distal tip 212 may be included within the distal portion 216. In the illustrated embodiment, the secondary mechanical layer 250 includes a closed distal end. However, in other embodiments, the secondary mechanical layer 250 may include an open distal end.

The secondary mechanical layer 250 may be formed of a plastic, elastomeric or metallic material. In some embodiments, the secondary mechanical layer 250 may be structurally formed defining a lumen 251 within which the optical fiber 210 may be subsequently inserted. In other embodiments, the secondary mechanical layer 250 may be formed onto the optical fiber 210, such as sprayed onto, over-molded onto, or otherwise applied to the optical fiber 210. In some embodiments, the secondary mechanical layer 250 may be attached to the optical fiber 210 so as to prevent longitudinal displacement of the secondary mechanical layer 250 with respect to the optical fiber 210. In some embodiments, the secondary mechanical layer 250, or more specifically a wall of the secondary mechanical layer 250, may define a fluid barrier. The structure of the secondary mechanical layer 250 may be defined so as to allow the optical fiber 210 to flex/bend in accordance with the shape sensing of the optical fiber 210.

In some embodiments, the assembly 200 may include a buffering substance 255 disposed within the lumen 251 so as to occupy the annular space between the optical fiber 210 and the secondary mechanical layer 250. The buffering substance 255 may further inhibit/prevent damage to the distal tip 212 during use of the optical fiber 210 in combination with the expansion 230 and the secondary mechanical layer 250. The substance may include an elastomeric material or a fluid. In some embodiments, the buffering substance 255 may be electrically conductive and may be electrically coupled with the expansion 230.

In some embodiments, the assembly 200 may include electrically conductive elements 222, such as wires, or traces, for example, extending along the optical fiber 210. The electrically conductive elements 222 may be electrically coupled with the expansion 230. As such, the electrically conductive elements 222 may be configured to receive electrical signals from the electrically conductive expansion 230 and transport the electrical signals proximally along the optical fiber assembly 200. As discussed above, the buffering substance 255 and/or the secondary mechanical layer 250 may be electrically conductive, and therefore, either or both of the buffering substance 255 and the secondary mechanical layer 250 may be configured to transport the electrical signals proximally along the optical fiber assembly 200.

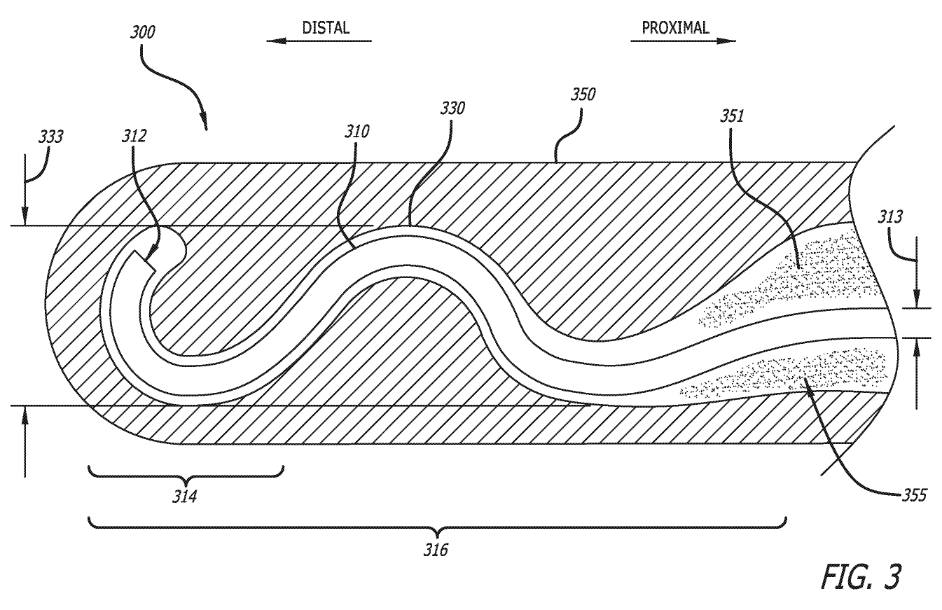

FIG. 3 is an illustration of a third embodiment of a fiber optic assembly 300. The fiber-optic assembly (assembly) 300 includes an optical fiber 310 and a secondary mechanical layer 350 extending around a circumference of the optical fiber 310. The optical fiber 310 defines a distal tip 312, where the distal tip 312 defines the distal most end of at least a subset of optical operations of the optical fiber 310. In other words, at least one optical functionality of the optical fiber 310 extends to the distal tip 312. The optical fiber 310 may optionally be configured for shape sensing.

The optical fiber 310 is disposed with a circuitous path 330 along a distal section 314 of the optical fiber 310 where the distal section 314 encompasses the distal tip 312. In the illustrated embodiment, the distal section 314 extends distally in relation to the distal tip 312.

The secondary mechanical layer 350 is also configured to inhibit/prevent damage to the distal tip 312 during use of the optical fiber 310 in combination with the expansion 330. The secondary mechanical layer 350 may define an enhanced structural integrity to the optical fiber, such as a resistance to crushing, abrasion, or kinking, for example. The secondary mechanical layer 350 may be disposed along a distal portion 316 of the optical fiber 310. The distal portion 316 generally extends proximally away from the distal tip 312. In some embodiments, the distal portion 316 may extend a proximally away from the distal tip 312 a distance of about 1 mm, 2 mm, 4 mm, 8 mm, 16 mm, or more. The secondary mechanical layer 350 may also extended distally away from the distal tip 312. As such, the distal section 314 may be included within the distal portion 316. In the illustrated embodiment, the secondary mechanical layer 350 includes a closed distal end. However, in other embodiments, the secondary mechanical layer 350 may include an open distal end.

The secondary mechanical layer 350 defines the circuitous path 330. In the illustrated embodiment, the circuitous path 330 defines a diameter (lateral dimension) 333 of the optical fiber 310 along the distal section 314, where the diameter 333 is greater than the diameter 313 of the optical fiber 310. In other embodiments, the circuitous path 330 may be substantially straight, such that the diameter 333 is similar to the diameter 313. In the illustrated embodiments, the circuitous path 330 may define a serpentine shape. In other embodiments, the circuitous path 330 may define a different shape, such as a spiral, a coil, a loop, a J-shape, or any combination thereof, for example. In some embodiments, the circuitous path 330 may be disposed within a single plane, i.e., a single lateral dimension with respect to a longitudinal axis of the secondary mechanical layer 350. In other embodiments, the circuitous path 330 may extend in both lateral dimensions with respect to the longitudinal axis.

The secondary mechanical layer 350 may be formed of a plastic, elastomeric, or metallic material. In some embodiments, the secondary mechanical layer 350 may be structurally formed defining a lumen 351 which defines the circuitous path 330 along the distal section 314. In some embodiments, the secondary mechanical layer 350 may be attached to the optical fiber 310 so as to prevent longitudinal displacement of the secondary mechanical layer 350 with respect to the optical fiber 310.

The secondary mechanical layer 350 may be formed of a single component or multiple components. For example, secondary mechanical layer 350 may be formed of a solid material having the circuitous path 330 formed therein, according to some embodiments. In other embodiments, the secondary mechanical layer 350 may include (i) an outer wall, such as a tube or a coil, for example, defining the lumen 351 and (ii) a buffering substance 355 disposed within the lumen 351, where the circuitous path 330 extends through the buffering substance 355. The buffering substance 355 substance may include an elastomeric material. In some embodiments, the assembly 300 may be assembled by first defining the circuitous path 330 and then inserting the optical fiber 310 therein. In other embodiments, the assembly 300 may be assembled by (i) forming a circuitous shape of the optical fiber 310, (ii) placing the circuitous shape of the optical fiber 310 in the lumen 351, and (iii) filling the lumen 351 with the buffering substance 355.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A fiber-optic assembly for insertion in a patient, comprising:
a shape sensing optical fiber including a distal section, the distal section comprising:
an optical fiber tip; and
an expansion configured to inhibit damage of the optical fiber tip, wherein the expansion includes:
a proximal end proximal of the optical fiber tip,
a distal end distal of the optical fiber tip, and
a diameter greater than a diameter of the shape sensing optical fiber; and
a mechanical layer surrounding the distal section of the optical fiber.

2. The fiber-optic assembly according to claim 1, wherein:
the optical fiber includes a plurality of optical fiber cores;
one or more of the plurality of optical fiber cores includes a plurality of sensors distributed along at least the distal portion of the optical fiber; and
the plurality of sensors are configured to project reflected light signals proximally along the optical fiber to indicate a shape of the optical fiber.

3. The fiber-optic assembly according to claim 1, wherein the mechanical layer includes a closed distal end.

4. The fiber-optic assembly according to claim 1, wherein the expansion extends beyond an open end of the mechanical layer.

5. The fiber-optic assembly according to claim 1, further comprising a substance disposed in the mechanical layer, the substance providing a mechanical buffer between the optical fiber and the mechanical layer.

6. The fiber-optic assembly according to claim 5, wherein the substance includes an elastomeric material.

7. The fiber-optic assembly according to claim 5, wherein the substance includes a fluid.

8. The fiber-optic assembly according to claim 1, wherein the expansion is integrally formed with the optical fiber.

9. The fiber-optic assembly according to claim 1, wherein the expansion is formed of an elastomeric material attached to the optical fiber.

10. The fiber-optic assembly according to claim 1, wherein the expansion is electrically conductive.

11. The fiber-optic assembly according to claim 1, wherein the mechanical layer is electrically conductive.

12. The fiber-optic assembly according to claim 1, wherein the expansion is electrically coupled with the mechanical layer.

13. The fiber-optic assembly according to claim 1, wherein the mechanical layer is coupled to the optical fiber proximal of the optical fiber tip.

14. The fiber-optic assembly according to claim 1.

15. An elongate medical device assembly, comprising:
the fiber-optic assembly according to claim 1; and
a catheter, a stylet, a probe, or a guidewire associated with the fiber-optic assembly.

16. The elongate medical assembly according to claim 15, further comprising a plurality of electrically conductive elements extending along the optical fiber, wherein the expansion is formed of an electrically conductive material to define an electrode of the elongate medical device, and wherein the electrode is coupled with at least one of the plurality of electrically conductive elements.
